(19) United States
(12) Patent Application Publication
(10) Pub. No.: US 2017/0369517 A1
(43) Pub. Date: Dec. 28, 2017

(54) COMPOSITIONS, METHODS AND SYSTEMS FOR DERIVATION OF USEFUL PRODUCTS FROM AGRICULTURAL BY-PRODUCTS

(71) Applicants: ALLIANCE FOR SUSTAINABLE ENERGY, LLC., Golden, CO (US); GENERAL MILLS, INC., Minneapolis, MN (US)

(72) Inventors: Joseph Shekiro, III, Arvada, CO (US); Nate Lukecart, Wauwatosa, WI (US); Christopher John Scarlata, Wheat Ridge, CO (US); Richard T. Elander, Evergreen, CO (US); Bernhard Van Lengerich, Minneapolis, MN (US); David Templeton, Golden, CO (US)

(21) Appl. No.: 15/534,970

(22) PCT Filed: Dec. 9, 2015
(86) PCT No.: PCT/US15/64772

§ 371 Date: Jun. 9, 2017

Related U.S. Application Data
(60) Provisional application No. 62/091,389, filed on Dec. 12, 2014.

(51) Int. Cl. C07H 1/08; C07H 3/06; C08B 37/00; C08H 8/00

(52) U.S. Cl. CPC C07H 1/08; C08H 8/00; C07H 3/06; C08B 37/0057

(57) ABSTRACT

Embodiments of the present invention report compositions, systems and methods for obtaining xylo-oligosaccharide-rich extracts from agricultural by-product streams. In certain embodiments, compositions and methods are directed to producing xylo-oligosaccharide-rich extracts with increased amounts and/or concentrations of degrees of polymerization (DP) of 3 or greater (DP3+). In other embodiments, compositions and methods relate to the production of products containing high soluble fiber, for example, xylo-oligosaccharides of DP3 or greater, from oat hull by-products. In yet other embodiments, xylo-oligosaccharides-rich extracts derived from oat hulls can be further processed to generate useful liquids for example, liquids containing soluble solids and powdered sweeteners and other useful consumer products.

### RELATED APPLICATIONS

This PCT application claims the benefit of U.S. Provisional Patent Application Ser. No. 62/091,389, filed Dec. 12, 2014. This application is incorporated herein by reference in its entirety for all purposes.

### GOVERNMENT FUNDING

This invention was made with government support under Contract No. DE-AC36-08GO28308 between the United States Department of Energy and Alliance for Sustainable Energy, LLC, the Manager and Operator of the National Renewable Energy Laboratory (NREL), and under CRADA No. CRD-12-483 between General Mills Operations, LLC, and NREL, operated for the United States Department of Energy. The Government has certain rights in this invention.

### FIELD

Embodiments of the present invention report compositions, systems and methods for obtaining xylo-oligosaccharide-rich extracts from agricultural by-product streams. In certain embodiments, compositions and methods are directed to producing xylo-oligosaccharide-rich extracts, including xylo-oligosaccharide-rich extracts with greater overall amounts and/or concentrations of xylo-oligosaccharides having degrees of polymerization (DP) of 3 or greater (DP3+). These DP3+ enriched compositions are enriched with DP3+ when compared to the total amount of xylans such as xylo-oligosaccharides having a DP of 2 or other sugars (xylose) with a DP of 1. In other embodiments, these xylo-oligosaccharides-rich extracts can be further processed to generate useful liquids for example, liquids containing soluble solids over a range of concentrations and amounts, or dried into a powdered sweetener, for example.

### BACKGROUND

There has been an increase in both the cost and volatility of basic food commodities, and such volatility in global food prices contributes significantly to economic uncertainty, especially in socio-economically vulnerable areas of the world. In addition, world distribution of agricultural endowment is uneven, and this has increased market volatility for basic food commodities, including certain carbohydrate and fiber sources. As global populations grow, certain regions of the world would greatly benefit from higher agricultural utilization of crop biomass or agricultural residue as an alternative source of nutrition, as well as reducing by-product materials and wastes. In addition, other sources of fiber are sought after that can compete with current products providing more efficiently produced and cost effective alternatives. Thus, there is a need to develop alternate sources and methods for converting biomass by-product into sources of food, food ingredients, substitutes and supplements.

### SUMMARY

Embodiments of the present disclosure report compositions, systems and methods for obtaining xylo-oligosaccharide-rich extracts from agricultural by-product streams. In certain embodiments, compositions and methods are directed to producing xylo-oligosaccharide-rich extracts, including xylo-oligosaccharide-rich extracts with greater overall amounts and/or concentrations of xylo-oligosaccharides having degrees of polymerization (DP) of 3 or greater (DP3+). These DP3+ enriched compositions are enriched with DP3+ when compared to the total amount of xylans, such as xylo-oligosaccharides having a DP of 2 or other sugars (xylose) with a DP of 1 (also referred to by one skilled in the relevant art as xylobios or monomeric xylose, respectively). In other embodiments, compositions and methods relate to the production of food ingredients containing significant levels of soluble fibers, for example, xylo-oligosaccharides of DP3 or greater, derived from oat hulls as described herein. Production of these products can be performed at reduced complexity and intensity resulting in xylo-oligosaccharide-rich extracts produced more efficiently and cost effectively.

In yet other embodiments, these xylo-oligosaccharides-rich extracts can be further processed to generate useful liquids, for example, liquids containing soluble solids over a range of concentrations or amounts, or dried into a powdered sweetener.

Certain compositions targeted by the methods disclosed herein include carbohydrates or polysaccharides, rich in non-digestible components, where the composition is enriched in polysaccharides DP3 or greater. In accordance with the embodiments, the enriched compositions contain polysaccharides having a high degree of solubility, and/or compositions that are considered soluble dietary fibers. Further, these xylo-oligosaccharide-rich extracts, as referred to above, include, but are not limited to, mixtures of monosaccharides and polysaccharides containing a variety of different monomers that include but are not limited to, for example, glucose, xylose, and arabinose monomer units. In certain embodiments, these compositions are enriched in xylan based polymers or Xylo-oligomers of DP3 or greater.

In certain embodiments, xylo-oligosaccharide-rich extracts can be used to generate syrups (rich in non-digestible components), fiber-rich ingredients for foods, powders or glucose-rich syrups each derived from oat-hull by-products and/or biomass feedstock as provided herein. It is contemplated that any of these compositions can be used as an ingredient in food products as a supplement or as a replacement ingredient.

Another aspect of the present invention includes xylo-oligosaccharide extract compositions derived from various biomass feedstock, such as oat hull feedstock. In accordance with these aspects, compositions can include xylo-oligosaccharides with DP3 or greater present in the extract at a concentration equal to or greater than the concentration of xylo-oligosaccharides with DP2 and/or DP1 (also referred by one skilled in the relevant art to as xylobios and monomeric xylose, respectively). In some embodiments, the amount of the xylo-oligosaccharides with DP3 or greater present in the xylo-oligosaccharide-rich extract is at least 15% of total xylan (including monomeric xylose forms) present in the oat hull derived xylo-oligosaccharide-rich extract. In some embodiments, the concentration of xylo-oligosaccharides with DP3 or greater compared to the concentration of xylo-oligosaccharides with DP2 and/or monomeric xylose of DP1 is at least about a 1 to 1 ratio, at least about a 1.5 to 1 ratio, at least about a 2 to 1 ratio, at least about a 2.5 to 1 ratio, at least about a 3 to 1 ratio, or at least about a 4 to 1 ratio, etc. In some embodiments, the amount of DP3+ in a xylo-oligosaccharide-rich extract is about 15.0% to about 80.0% of the total xylan (whereby total xylan includes monomeric xyloses) present in the xylo-oligosaccharide-rich extract. In other embodiments, the amount of DP3+ in a xylo-oligosaccharide-rich extract is about 20.0% to about 80.0% of the total xylan present in the xylo-oligosaccharide-rich extract, or about 25.0% to about 70.0% of the total xylan present in the xylo-oligosaccharide-rich extract or other similar amount.

Embodiments of the present invention also provide methods for obtaining or concentrating or fractionating out xylo-oligosaccharides with DP3 or greater from various biomass feedstock, such as oat hull feedstock. Certain embodiments include methods for providing oat hulls, treating the oat hulls to remove undesirable compounds (e.g., inorganic compounds, sulfur-containing compounds, chloride, sodium, phosphorus, magnesium, starches and the like), cooking the oat hulls at a temperature about 170° C. to about 220° C. for about 1 minute and about 30 minutes, and separating insoluble solid fractions from soluble solid fractions. In accordance with these embodiments, the soluble solid fractions include, but are not limited to, an xylo-oligosaccharide-rich extract that includes xylo-oligosaccharides with DP3 or greater. Additionally, xylo-oligosaccharide-rich extracts produced according to the disclosed herein can include xylo-oligosaccharides with DP3 or greater at a concentration equal to or greater than the concentration of xylo-oligosaccharides with DP2 or sugars with DP1 (e.g., monomeric xylose). Alternatively, the amount of the xylo-oligosaccharides with DP3 or greater in a xylo-oligosaccharide-rich extract from oat hulls as disclosed herein can be at least 15% of total xylan (including monomeric xylose) present in the xylo-oligosaccharide-rich extract.

Another aspect of the methods disclosed herein can include milling the oat hulls prior to cooking. In certain embodiments, milling the oat hulls can include milling the oat hulls to about 1 millimeter and about 10 millimeters in diameter. In some embodiments, the method can further include washing the oat hulls with an aqueous solution and draining off the aqueous solution, for example, to remove undesirable compounds (e.g., chloride, magnesium, sodium, sulfates, other unwanted minerals, and the like). In other embodiments, methods may include diluting the oat hulls in an aqueous solution after cooking but prior to separating the insoluble solids fraction from the soluble solids fraction and any liquid. In other embodiments, methods can include washing the insoluble solids fraction prior to subjecting the insoluble solids fraction to additional cooking and further fractionating out additional xylo-oligosaccharides with DP3 or greater.

Another aspect of the method can include cooking the oat hulls at a temperature between about 170° C. to about 210° C. for about 1 to about 30 minutes. In other embodiments, methods can include cooking the oat hulls at a temperature between about 180° C. to about 200° C. for about 1 to about 20 minutes, 180° C. to about 195° C. for about 2 to about 15 minutes, 185° C. to about 195° C. for about 2 to about 10 minutes, and 190° C. for about 4 to about 10 minutes.

Other embodiments can include lightening the color of a xylo-oligosaccharide-rich extract enriched in DP3 or greater for example to add to a consumable product. In some embodiments, methods can include forming a powder from the xylo-oligosaccharide-rich extract. In other embodiments, methods can include adding additional agents to xylo-oligosaccharide-rich extract prior to adding it to a consumable food product.

### BRIEF DESCRIPTION OF THE DRAWINGS

The following drawings form part of the present specification and are included to further demonstrate certain embodiments. Some embodiments may be better understood by reference to one or more of these drawings alone or in combination with the detailed description of specific embodiments presented.

FIG. 1 is a schematic representation of certain embodiments disclosed herein regarding the removal and processing of oat hulls into a xylo-oligosaccharide-rich extract.

FIG. 2 is a schematic representation of certain embodiments disclosed herein regarding processing of oat hulls into a xylo-oligosaccharide-rich extract and analyses thereof.

### DEFINITIONS

As used herein, “a” or “an” may mean one or more than one of an item.

As used herein the specification, “subject” or “subjects” may include but are not limited mammals such as humans or mammals, domesticated or wild, for example dogs, cats, other household pets (e.g., hamster, guinea pig, mouse, rat), ferrets, rabbits, pigs, horses, cattle, prairie dogs, or zoo animals.

As used herein, “about” or “approximately” can mean plus or minus ten percent.

As used herein, “xylan” can mean polymers of individual xylose monomers (a 5-carbon sugar) connected with 1,4-β bonds to form polysaccharides, and/or monomeric xylose and polymeric xylose constituents or subcomponents of hemicellulose. For example, xylans can include, but are not limited to, arabinoxylan, glucuronoxylan, xyloglucan and xylans originating from these three agents, or any combinations thereof.

As used herein, “xylo-oligosaccharide” or “xylo-oligosaccharides” or “XOS” can mean water soluble fractions of polymers of the sugar xylose wherein the degrees of polymerization range from about DP2 to about DP20.

As used herein, “degrees of polymerization” or “DP” can mean an average number of base units per molecule if the molecules are composed of regularly repeating units, or as an average number of monomeric units (e.g., mers) per molecule. For example, a xylo-oligosaccharide of DP3 or greater can include, but is not limited to, three or more monomeric xylose units.

As used herein, “slurry” can mean a mixture of insoluble solids, soluble solids, and liquid that can be obtained from grain by-products and biomass feedstock material, such as oat hull by-products and oat hull biomass feedstock material.

As used herein, “xylo-oligosaccharide extract” or “XOS extract” or “xylo-oligosaccharide-rich extract” can mean a mixture or composition including, but not limited to, DP3 or greater xylo-oligosaccharides (DP3+). For example, “xylo-oligosaccharide extract” or “XOS extract” or “xylo-oligosaccharide-rich extract” can be obtained from a liquid and/or soluble solid fraction of oat hull slurry.

### DETAILED DESCRIPTION

In the following sections, various exemplary compositions and methods are described in order to detail various embodiments. It will be obvious to one skilled in the art that practicing the various embodiments does not require the employment of all or even some of the details outlined herein, but rather that concentrations, times and other details may be modified through routine experimentation. In some cases, well-known methods or components have not been included in the description.

Embodiments of the present invention provide for compositions and methods for obtaining xylo-oligosaccharide-rich extracts from agricultural by-product and/or waste streams. In certain embodiments, compositions and methods are directed to producing xylo-oligosaccharide-rich extracts, including xylo-oligosaccharide-rich extracts with greater overall amounts and/or concentrations of xylo-oligosaccharides having degrees of polymerization (DP) of 3 or greater (DP3+). These DP3+ enriched compositions are enriched with DP3+ when compared to the total amount of xylans in an extract, such as xylo-oligosaccharides having a DP of 2 or less (e.g., DP of 1, which is also referred to as xylose).

In other embodiments, compositions and methods relate to the production of food ingredients containing significant levels of soluble fibers, including, for example, xylo-oligosaccharides of DP3 or greater, derived from oat hulls, as described herein. Production of these food ingredients can be done using less complex methods compared to conventional or known methods, thus resulting in more efficient, cost effective production of xylo-oligosaccharide-rich extracts. In yet other embodiments, these xylo-oligosaccharides-rich extracts can be processed to generate useful liquids, including, for example, liquids containing soluble solids over a range of concentrations and amounts, or these xylo-oligosaccharides-rich extracts can be processed into powdered compositions (e.g., sweeteners). It is contemplated herein that any of these compositions can be used as a main ingredient in a food product, as well as a supplement or a replacement ingredient.

Embodiments of the present invention include compositions, systems and methods for obtaining and using xylo-oligosaccharide-rich extracts from oat hull by-product streams and feedstock. In certain embodiments, compositions and methods are directed to producing xylo-oligosaccharide-rich extracts that include xylo-oligosaccharide-rich extracts with greater overall amounts and/or concentrations of xylo-oligosaccharides of DP3 or greater (DP3+). These DP3+ enriched compositions are enriched with DP3+ when compared to the total amount of xylans such as xylo-oligosaccharides having a DP of 2 or other sugars (monomeric xylose) with a DP of 1. In other embodiments, compositions and methods relate to the production of food ingredients containing significant levels of soluble fibers, for example, xylo-oligosaccharides of DP3 or greater, derived from oat hulls as described herein.

There exists a growing consumer interest in incorporating additional dietary fibers and prebiotics into a range of products to promote health. There are several issues that have created barriers to broad incorporation of dietary fiber in food products that include negative organoleptic properties and that these compositions are expensive to produce. Traditional insoluble dietary fibers are generally inexpensive but degrade product quality. Soluble fibers are generally easier to incorporate without loss of quality but are significantly more expensive. The instant application provides for cost effective substitutes and additives as well as generating useful products from what would otherwise be by-products (or unused wastes).

Biomass feedstock and/or agricultural by-products can be a source of non-digestible carbohydrates (e.g., fiber). Such non-digestible carbohydrates can be added to many consumer food products as a fortificant. For example, dietary fibers that are highly soluble are amenable to incorporation into consumer food products without compromising hedonic qualities such as taste or texture. Embodiments herein provide for materials and methods for producing and/or extracting various carbohydrates and polysaccharides having beneficial qualities, including but not limited to, polysaccharides rich in non-digestible components, polysaccharides with DP3 or greater, polysaccharides having a high degree of solubility, carbohydrates considered dietary fiber, carbohydrates considered soluble dietary fibers, and the like. Xylo-oligosaccharide-rich extracts produced using methods disclosed herein can include polysaccharides composed of a variety of different monomers, including but not limited to, glucose, xylose, and arabinose monomer units. In certain embodiments, extracts are enriched with xylo-oligosaccharides with DP3 or greater, as compared to xylo-oligosaccharides with DP2 and/or DP1 (e.g., monomeric xylose) where extracts having enriched DP3+ can serve as a source of dietary fiber.

Hemicellulose, a source of xylo-oligosaccharides, is a structurally diverse cell wall polymer found in woody and annual plants. Hemicellulose is comprised of a 1,4-β-D-xylopyranosyl backbone with various side chains linked to xylopyranosyl, arabinofuranosyl, 4-O-methyl-D-glucuronopyranosyl, D-galactopyranysol, or D-glucurono pyranosyl units and acetyl linkages. The 1,4-β-D-xylopyranosyl backbone of hemicellulose is often referred to as xylan. Structural carbohydrates such as xylan can be defined by the empirical methods used to quantify those components in a biomass sample. For example, methods such as those disclosed in Sluiter, A., et al., (Determination of Structural Carbohydrates and Lignin in Biomass (2012), NREL/TP-510-42618), widely used by those skilled in the art to quantify xylan in biomass.

Extracts produced using the methods of the present invention can be heterogeneous mixtures that include xylose and/or polymers of xylose, glucose and/or polymers of glucose, arabinose and/or polymers of arabinose, and galactose and/or polymers of galactose. Xylan in oligomeric forms, referred to as xylo-oligosaccharides or XOS, are considered dietary fibers for the purposes of food products or ingredients. XOS have degrees of polymerization (DP) in the range of about 3 to about 7 and are generally soluble. In some aspects, methods of the present disclosure can be used to produce extracts having XOS concentrations of DP3 or greater between about 50% and about 80% of the extract. In some aspects, the concentrations of XOS of DP3 or greater can be greater than the concentrations of XOS of DP2 or DP1 in a given extract. In some aspects, extract produced using the embodiments herein can have reduced amounts of undesirable components. For example, methods that rely predominantly on chemical technologies (e.g., acid hydrolysis) for treating lignocellulosic biomass often lead to the increased release of undesirable compounds or agents, such as furfural. In contrast, the methods herein can produce extracts with reduced amounts of furfural and other undesirable products as compared to other methods of extractions (e.g., chemical extraction methods).

In certain embodiments, dietary fiber sources, reported to promote beneficial microbial growth in the large intestine, can be generated by methods disclosed herein using grain by-products or residues. Certain embodiments are directed toward producing a low cost source of dietary fiber from oat hulls. For example, xylo-oligosaccharides (XOS) are a potential source of dietary fiber and have been reported to have beneficial pre-biotic and other dietary effects. XOS having other beneficial health properties can be extracted using the methods of the present invention. For example, slurry produced using these methods can include XOS with various substituents that enhance their nutritional value (e.g., enhance prebiotic characteristics). For example, XOS having increased acetyl and uronic substituents can be extracted using the methods disclosed herein.

Another aspect of the present disclosure includes xylo-oligosaccharide extract compositions derived from various biomass feedstock, such as oat hull feedstock. In accordance with these aspects, compositions can include DP3+ xylo-oligosaccharides at a concentration equal to or greater than the concentration of xylo-oligosaccharides with DP2 and/or DP1 (monomeric xylose). In some embodiments, the amount of the xylo-oligosaccharides with DP3 or greater present in the xylo-oligosaccharide-rich extract is at least 15% of total xylan (including monomeric xyloses) present in the oat hull derived xylo-oligosaccharide-rich extract. In some embodiments, the concentration of xylo-oligosaccharides with DP3 or greater compared to the concentration of xylo-oligosaccharides with DP2 and/or DP1 (monomeric xylose) is in an equal ratio, at least about a 1.5 to 1 ratio; at least about a 2 to 1 ratio, at least about a 2.5 to 1 ratio, at least about a 3 to 1 ratio, at least about a 4 to 1 ratio, or higher ratio. In some embodiments, the amount of DP3+ in a xylo-oligosaccharide-rich extract is about 15.0% to about 90.0% of the total xylan present in the xylo-oligosaccharide-rich extract. In other embodiments, the amount of DP3+ in a xylo-oligosaccharide-rich extract is about 20.0% to about 80.0% of the total xylan present in the xylo-oligosaccharide-rich extract, about 25.0% to about 70.0% of the total xylan present in the xylo-oligosaccharide-rich extract, or about 30.0% to about 60.0% of the total xylan present in the xylo-oligosaccharide-rich extract. These amounts can vary depending on the starting oat hull material (total amount of DP3+ at the start of an extraction process) as well as the end-point selected for processing of oat-hull by-products.

Some aspects of the present disclosure can include use of XOS-enriched extract as a component of various food products. XOS-enriched extract can be added to consumable products for example, to increase the amount of soluble fiber (e.g., carbohydrates that are DP3 and greater and at least non-digestible) in the consumable. In accordance with these aspects, these additions can increase overall nutritional value of the consumable. XOS enriched extract can be an additional component of a food product and/or XOS enriched extract can replace another component of the food product. For example, XOS enriched extract can be an added component of food products that include, but are not limited to, energy bars, breakfast bars, ice creams, beverages, energy drinks, cereals, breads, and other processed foods or freshly prepared foods. XOS enriched extract can also be a substitute for currently available sources of fiber, such as inulin, that are added to various food products. In some aspects, XOS enriched extract produced using methods disclosed herein can be more efficiently produced and more cost-effective than other available sources, such as inulin (e.g., chicory root extract). In other aspects, XOS enriched extract can be concentrated to form edible syrup that can be added to various food products.

One method for obtaining xylo-oligosaccharides with degrees of polymerization (DP) 3 or greater from oat hulls can include, but is not limited to:

providing oat hulls;

treating the oat hulls to remove undesirable compounds;

cooking the treated oat hulls at a temperature between about 170° C. to about 220° C. for about 1 minute to about 30 minutes; and

separating an insoluble solids fraction from a soluble solids fraction, wherein the soluble solids fraction is an xylo-oligosaccharide-rich extract having higher levels of xylo-oligosaccharides with DP3 or greater than found in a starting oat hull byproduct. Oat hulls of this method can include, but are not limited to oat-hull byproducts and/or oat hull feedstock.

In other embodiments, methods can further include separating liquids from the solids after cooking the drained milled oat hulls in order to obtain usable XOS slurry compositions. In accordance with these methods, unmodified oat hulls or oat hulls that have been milled and/or drained can be cooked at a temperature of between about 175° C. to about 200° C. Other embodiments can include cooking oat hulls at a temperature of about 175° C. to about 200° C. for about 1 to about 14 minutes. The timing and temperature can be based on reducing the amounts of DP1 and DP2 sugars in the extract. In yet other embodiments, cooking oat hulls can include cooking for about 3 to about 9 minutes at a temperature of between about 185° C. to about 200° C. Certain embodiments concern cooking oat hulls for about 3 to about 9 minutes at a temperature of about 190° C. to 195° C.; or for about 4-8 minutes at a temperature of about 190° C. In one embodiment, the oat hulls are cooked for about 6-8 minutes at a temperature of about 190° C.

Other methods of preparing edible xylo-oligosaccharide-rich extract can include, further demineralizing the xylo-oligosaccharide-rich extract and/or removing the odor from the xylo-oligosaccharide-rich extract. Oat hull derived xylo-oligosaccharide-rich extract produced by these methods can be filtered, if desired, to remove unwanted agents. If desired, the color of xylo-oligosaccharide-rich extracts can be altered, for example, to make it more appealing.

In other embodiments, oat hulls can be milled prior to cooking. In certain embodiments, oat hulls can be milled to about 1 millimeter to about 20 millimeters or 1 millimeter to about 10 millimeters in size. In some embodiments, oat hulls can be treated by washing with an aqueous solution and draining off the aqueous solution to remove minerals (e.g., chloride, sodium, sulfates, and the like). The oat hulls can also be diluted in an aqueous solution after cooking but prior to separating the insoluble solids fraction from the soluble solids fraction. In some embodiments, the insoluble solids fraction can be washed prior to subjecting the insoluble fraction to additional cooking and separating steps to obtain remaining/uncollected xylo-oligosaccharide-rich extracts with DP3 or greater, therefore increasing the amount of product recovered from the oat hull byproduct or feedstock.

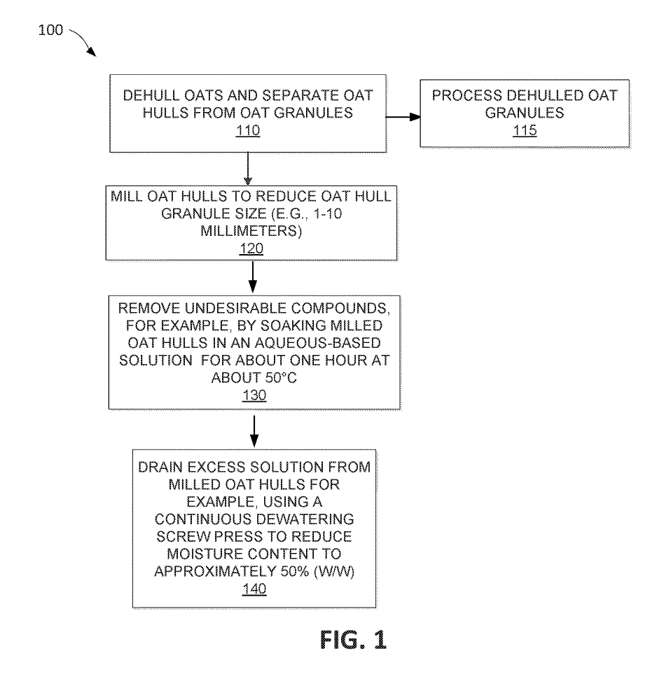

Embodiments of the present invention include methods for removing the predominantly lignocellulosic portions (e.g., hulls for example of oats, barley and rice) from various plants, such as wheat, corn, barley and rice, and using these lignocellulosic portions as a feedstock to produce consumable food products. For example, as shown in FIG. 1, the present invention includes a method for removing and processing oat hulls 100. Oat hulls are dehulled and separated from the groat (block 110). The groat, or dehulled oat granules, can be processed and used to make various consumable food products (block 115). Oat hulls are milled to reduce overall oat granule size to between about 1 millimeters and about 10 millimeters (block 120). To remove undesirable compounds, an aqueous-based solution can be added to the milled oat hulls to obtain about a 10% w/w mixture of the milled oat hulls (e.g. enough aqueous solution is added to make 1 part aqueous solution to 9 parts milled oat hulls to make the oat hull mixture), and the mixture of milled oat hulls and the aqueous-based solution is soaked for about 1 hour at an elevated temperature (e.g., 50° C.) (block 130). Undesirable compounds can include, but are not limited to, inorganic compounds, sulfur-containing compounds, chloride, sodium, phosphorus, magnesium, starches and the like. In some aspects, the aqueous-based solution can be added at a ratio of about 5% and about 25% of the milled oat hulls, and soaking can be from about 30 minutes to about 3 hours at a temperature between about 15° C. and about 100° C., depending on the various conditions of the process, as would be appreciated by one of skill in the art and based on the present disclosure.

Additionally or alternatively, alkaline extraction can be performed on a mixture of milled oat hulls in the aqueous-based solution to enhance pretreatment processing. In some aspects, dilute sodium hydroxide (e.g., 0.4% w/w) can be added to the milled oat hull mixture and continuously mixed and heated indirectly at about 80° C. for about 2 hours. Other alkaline solutions can be used as appreciated by one of skill in the art and based on the present disclosure. In some aspects, the milled oat hull mixture can be dewatered until the moisture content is between about 25% and about 75% w/w of the milled oat hulls, if desired.

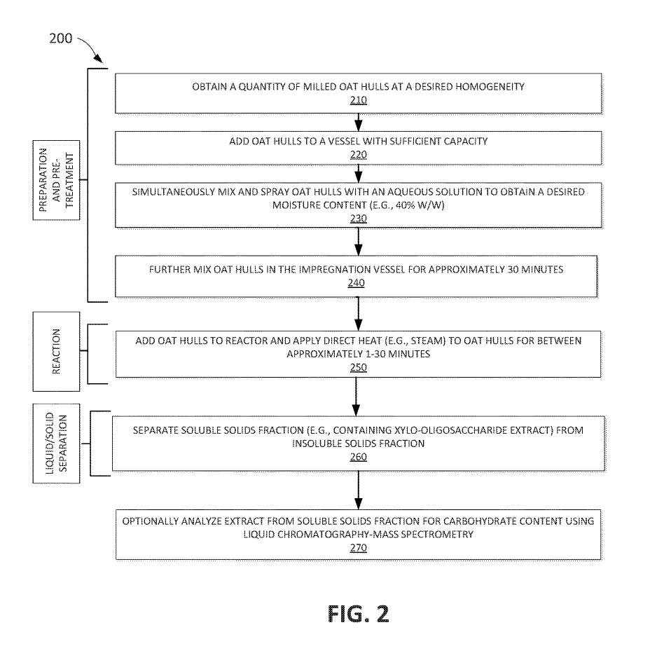

Embodiments of the present disclosure also include methods for extracting components from predominantly lignocellulosic portions (e.g., hulls of oats, rice and barley for example) of various plants, such as wheat, corn, barley and rice, for use as a feedstock to produce consumable food products. For example, as shown in FIG. 2, the present disclosure includes a method for extracting components from milled oat hulls 200. As an aspect of preparation and pretreatment, a quantity of milled oat hulls of a desired homogeneity is obtained (block 210). In some aspects, the milled oat hulls are obtained after being subjected to the processing methods described in FIG. 1. The milled oat hulls (e.g., 200-300 kilograms) are added to a vessel with sufficient capacity (e.g., 1000 liters) (block 220). Oat hulls are simultaneously mixed and sprayed with an aqueous-based solution to obtain desired moisture content, which can be in the range of about 20% to about 30%, to about 40% to about 50% to about 60% w/w of the oat hulls (block 230). In some aspects, the moisture content can be about 40% w/w. Oat hulls are further mixed in the impregnation vessel for a given amount of time, in some aspects between about 30 minutes and about 2 hours (block 240). In some aspects, the oat hulls can be stored in sealed containers at 4° C. until further processing (block 245).

In certain embodiment, regarding cooking or a reaction phase, oat hulls can be pre-heated indirectly (e.g., using steam jackets) at about 180° C. to about 200° C. for about 30 minutes to 2 hours, and in some aspects, for about 70 minutes. In another aspect of the reaction phase, oat hulls can be added to a reactor and direct heat (e.g., steam) can be applied to the oat hulls with or without mixing for about 1 minute and about 30 minutes (block 250).

As an aspect of liquid/solid separation, soluble solids fraction (e.g., the fraction that includes XOS as an XOS-enriched extract) is separated from the insoluble solids fraction (block 260). Oat hulls can be pressed using, for example, a hydraulic filter press at about 250 psi, and a vacuum-assisted recovery system is used to extract slurry containing various isolates from the milled oat hulls. In some aspects, oat hulls can be diluted with an aqueous-based solution to obtain about a 10% w/w mixture of the milled oat hulls, and centrifuged at about 1450 rpm to extract slurry containing various isolates from the milled oat hulls (block 380). In some aspects, pressing and vacuum-assisted recovery, and dilution and centrifugation, can be performed interchangeably and/or in succession. The XOS-enriched extract can be filtered using, for example, a pressure filter (e.g. 10 microns or other), and a solution can be added to preserve the slurry (e.g., sodium metabisulfate at about 200 ppm). In other aspects of the invention, the XOS extract can be analyzed using, for example, liquid chromatography-mass spectrometry, to determine the content and concentration of various isolates from the milled oat hulls (block 270), including, but not limited to, xylan, glucan, galactan, arabinan, and lignin, in various monomeric and oligomeric states.

In some embodiments, the methods of the present invention include extracting xylans from various sources of cellulosic biomass, including but not limited to, milled oat hulls. Using the methods of the present invention, an XOS-enriched extract of various components can be isolated from oat hull byproducts, in part, by subjecting the oat hulls to a reaction phase that includes heating the oat hulls to about 190° C. for about 6 minutes, followed by subjecting the oat hulls to an extraction process (e.g., centrifugation, vacuum-assisted extraction, filtering and the like). These conditions are sufficient to facilitate hydrolysis to release xylans (and monomeric xyloses) contained within the cell walls of the oat hulls. In some aspects, enzymatic hydrolysis using an endoxylanase can be performed to enhance the extraction of the xylans (DP3 and greater as well as monomeric xyloses). In other aspects, the XOS-enriched extract and/or the soluble solids fraction of the XOS extract can be further refined using various means known in the art and based on the present disclosure (e.g., concentrating, purifying, evaporating, drying, and the like).

Some embodiments disclosed herein include a kit of one or more of the above referenced compositions. Kits contemplated herein can include a container for storing or transporting an XOS-enriched extract disclosed herein.

The following examples are included to demonstrate certain embodiments presented herein. It should be appreciated by those of skill in the art that the techniques disclosed in the examples that follow represent techniques discovered to function well in the practices disclosed herein. However, those of skill in the art should, in light of the present disclosure, appreciate that many changes can be made in the certain embodiments which are disclosed and still obtain a like or similar result without departing from the spirit and scope herein.

### EXAMPLES

### Example 1

### Oat Hull Processing

In certain exemplary methods, oat grains can be processed to separate the hull from the groat (FIG. 1). The oat hulls can be further washed to remove minerals (e.g., ash) by subjecting them to hot water washes and/or treatment with sodium hydroxide and heat. Additionally or alternatively, the oat hulls can be milled to produce granules of a desired size to facilitate downstream processing, including XOS extraction. If not directly processed, excess moisture can be drained from the oat hulls and the oat hulls can be stored in sealed containers under refrigeration for later processing.

In other exemplary methods, XOS production conditions from oat hulls are analyzed. In one method, starting oat hull by-product compositions are analyzed for target agents such as xylan and glucan and other useful products. Raw oat hull by-product extracts or other oat hull compositions are tested to assess whether they contain significant amounts of target components. In one example, oat hulls were provided in super sacks of approximately 220 kg. A series of samples were removed to generate a compositional profile and then evaluated for homogeneity. These sample compositions are reflected in Table 1. These compositions were a useful starting point for identifying conditions to produce an oat-hull derived high fiber extract. The xylan fraction accounted for about 30% of the total oat hull composition in these by-product samples. These values will likely vary.

TABLE 1
Example of a raw oat hull composition

| | Ash | Lignin | Glucan | Xylan | Galactan | Arabinan | Acetate | Total |
|---|---|---|---|---|---|---|---|---|
| | (%) | (%) | (%) | (%) | (%) | (%) | (%) | (%) |
| Average | 6.34 | 17.85 | 29.38 | 30.04 | 1.62 | 3.22 | 2.36 | 95.68 |
| Standard | 0.21 | 0.29 | 0.45 | 1.40 | 0.12 | 0.20 | 0.07 | 1.44 |
| Deviation | | | | | | | | |

In one example, the contents of a super sack of oat hulls (˜220 kg) were loaded into a mixing vessel (e.g., 1000 L). Then, the oat hulls were blended. In one example, the oat hulls were sprayed with water to achieve a target moisture content of about 30-50% (w/w). In this example, the oat hulls were sprayed with water to achieve a target moisture content of about 40%. The mixture was made agitated in the vessel for about 30 minutes prior to discharge. Upon removal from the vessel, oat hulls can be for additional processing.

### Example 2

### XOS Extraction

A single production experiment spanning six hours was executed in a continuous horizontal reactor system. The pretreatment reactor was in this example, indirectly pre-heated by steam jackets to 190° C. for 70 minutes before oat hulls started to be metered or fed into the system. Upon entering the reaction zone, oat hulls were heated to 190° C. by direct steam injection and fed through the system using a series of speed-controlled augers to target a reaction time of 6 minutes. Other times, temperatures and methods for performing a similar task are contemplated and known by those skilled in the art.

Analysis of the oat hull slurry compositions can indicate which conditions may maximize XOS production from oat hulls as well as minimize the presence of undesirable components, including, for example, furfural. Samples of process effluent were taken for analysis at various points throughout the run: for example 3X: 30 minutes and 3 hours after beginning collection of pretreated oat hulls, and approximately 30 minutes prior to reactor shut-down. Samples of pretreated oat hulls and the two reactor vent streams were taken at each of these time points.

In one example, eight initial reaction conditions were tested based upon previous findings that identified maximum conversion of oat hulls to xylo-oligomers occurred at about 200° C. for about 14 minutes. It was observed that little hydrolysis occurred below 190° C. Based on these observations, the conditions listed in Table 2 for the initial series pilot-scale screening experiments were analyzed. Other times, temperatures and methods for performing a similar task are contemplated and known by those skilled in the art.

Analysis of the samples in Table 2 indicated that time and temperatures were two conditions tested that could affect the ultimate content and concentrations of XOS. In one case, treatment at 190° C. for 6 minutes produced a product enriched for XOS. Because this was the lowest time and temperature tested, a second screening was executed, exploring reduced pretreatment intensity, as shown in Table 3.

TABLE 2
Run information and experimental conditions

| Sample Description | Temp (° C.) | Time (min) | pH |
|---|---|---|---|
| GMI120801-1 | 190 | 24 | 3.45 |
| GMI120801-2 | 190 | 18 | 3.52 |
| GMI120801-3 | 190 | 12 | 3.71 |
| GMI120801-4 | 190 | 6 | 3.92 |
| GMI120801-5 | 200 | 24 | 3.48 |
| GMI120801-6 | 200 | 18 | 3.42 |
| GMI120801-7 | 200 | 12 | 3.43 |
| GMI120801-8 | 200 | 6 | 3.55 |
| GMI120801-9 | 205 | 6 | 3.52 |

TABLE 3
Run information and experimental conditions

| Sample Description | Temp (° C.) | Time (min) | pH |
|---|---|---|---|
| GMI121114-1 | 180 | 6 | 3.3 |
| GMI121114-2 | 180 | 4.5 | 3.62 |
| GMI121114-3 | 185 | 6 | 3.65 |
| GMI121114-4 | 185 | 4.5 | 3.74 |
| GMI121114-5 | 190 | 6 | 3.67 |
| GMI121114-6 | 190 | 4.5 | 3.71 |

### Solid/Liquid Separation

Pretreated oat hulls were collected upon discharge from the pretreatment system at approximately 40% total solids by weight.

In one exemplary method, pretreated oat hulls were pressed for storage for example, to reduce any unnecessary dilution of the XOS solution. Samples were pressed using a hydraulic filter press at pressures of up to 250 psi. XOS solution was recovered via a vacuum-assisted liquor recovery system. After primary separation, the XOS solution was filtered via a 10 micron pressure filter and preserved.

Pretreated oat hulls were separated into solid and liquid fractions and levels of total and insoluble solids were measured, as well as the concentrations of agents such as sugars and organic acids. A subset of samples from the composition was selected for analysis of the solid fraction to complete a mass balance and calculate component yields. Reactor vent streams were analyzed to determine composition. Process conditions and stream flows were recorded by a data acquisition and control system (DACS).

Component yields (e.g., XOS yields) can be calculated using the following formula, other formulas can be used to estimate totals of the desired extracts:

$$XOS \% Yield = \frac{[ ( Total xylose - Monomeric xylose ) * Hydrolysate volume ]}{[ \begin{matrix}( Dry mass of feedstock ) * ( \% Xylan in feedstock ) * \\ ( Xylan to xylose conversion factor )\end{matrix} ]}$$

With the exception of the oligomer characterization described herein, samples were analyzed using standard techniques known in the art and provide in, for example, Sluiter, A., et al., (Determination of Structural Carbohydrates and Lignin in Biomass (2012), NREL/TP-510-42618); Sluiter, A. et al., (Determination of Ash in Biomass (2008), NREL/TP-510-42622); and Sluiter, A. et al., (Laboratory Analytical Procedure (LAP) (2008), NREL/TP-510-42619, publicly available), which disclose widely used methods for quantifying and analyzing the various components (e.g., xylans) in biomass.

Oligomers of these samples can be characterized, data not shown. In these exemplary methods, oligomers were characterized using liquid chromatography-mass spectrometry (LC-MS) to determine a distribution of degrees of polymerization for oligomers with 5 carbon monomer units. This distribution was applied to a previously quantified concentration of total xylo-oligomers to obtain concentrations of each component group. One skilled in the art would readily understand that there are many ways to characterize and analyze the samples.

In general, the lowest severity condition tested exhibited the best attributes: a comparatively high yield of long-chain xylo-oligomers, low yields of monomeric and dimeric xylose, and low amounts of degradation products. To validate further these results, subsequent analysis was performed using the favorable conditions analyzed to test decreasing temperature and/or reactor residence times. As indicated in Table 5, subsequent analysis confirmed the favorable results previously observed. In this example, pretreatment conditions that included treatment at 190° C. for 6 minutes yielded very positive results in terms of XOS production. One skilled in the art would readily understand that times and temperatures can vary widely depending on the type of instrumentation and methods used for cooking the oat hull slurries.

TABLE 5
Summary of XOS profiles

| | | | | Total | C5 | C5 | Total | C6 | | | |
|---|---|---|---|---|---|---|---|---|---|---|---|
| | | | Total | Sugars | DP1-2 | DP3+ | C5 | DP1 | C6 | Total | Organic |
| Sample | | | Dissolved | (% | (% | (% | (% | (% | Oligomeric | C6 (% | Acids |
| Description | Temp | Time | Solids | TDS) | TDS) | TDS) | TDS) | TDS) | (% TDS) | TDS) | (% TDS) |
| GMI120801-4 | 190 | 6 | 15% | 11.4% | 4.3% | 4.2% | 8.5% | 0.5% | 2.4% | 3.0% | 1.1% |
| GMI121114-1 | 180 | 6 | 12% | 10.9% | 3.3% | 3.9% | 7.2% | 0.7% | 1.4% | 2.1% | 0.8% |
| GMI121114-2 | 180 | 4.5 | 11% | 9.9% | 2.9% | 3.7% | 6.6% | 0.6% | 1.2% | 1.8% | 0.7% |
| GMI121114-3 | 185 | 6 | 13% | 11.9% | 2.7% | 6.0% | 8.7% | 0.5% | 1.5% | 2.0% | 0.9% |
| GMI121114-4 | 185 | 4.5 | 12% | 10.5% | 2.6% | 5.2% | 7.8% | 0.5% | 1.3% | 1.8% | 0.7% |
| GMI121114-5 | 190 | 6 | 15% | 13.0% | 2.9% | 7.1% | 9.9% | 0.5% | 1.7% | 2.2% | 1.0% |
| GMI121114-6 | 190 | 4.5 | 14% | 12.4% | 2.7% | 6.9% | 9.6% | 0.4% | 1.6% | 2.0% | 0.9% |

During oat hull processing and XOS extraction, samples can be assessed for consistency and performance characteristics. For example, results of XOS profile analysis of three samples taken during the course of a single processing event were analyzed (data not shown). The XOS profile characteristics were very consistent, including the ration of DP3+ to DP1 and DP2 five-carbon oligosaccharides. These results indicate that the XOS solution produced in these examples were consistent with those produced in prior testing. In this experiment, 55% of the recovered 5-carbon sugars were present as oligosaccharides with a degree of polymerization of 3 or greater. This exemplary process achieved a yield of approximately 30% of DP3+ XOS on a xylan basis. On an overall mass basis, 89 grams of DP3+ XOS were produced in pretreatment per kilogram of feedstock fed, before accounting for potential losses in solid-liquid separation and other downstream operations.

In solid-liquid separation, greater than 90% of the XOS solution was recovered by centrifugation at approximately 900 gravities. Dilution of the XOS solution to 10% total solids was used in order to facilitate mix and load the slurry into the centrifuge.

The execution of this verification experiment successfully demonstrated the repeatability of process performance, and provided in site prior to further scale-up. Approximately 55% of five carbon sugars solubilized in this process had degrees of polymerization of three or greater thus providing a reliable method for producing XOS enriched extract from oat hulls. Further, 30% of available xylan and 8.9% of overall mass were converted to the target product. With dilution of the pretreated oat hulls, centrifugation was an effective means of producing a clarified liquid stream from the process with minimal product loss.

In certain exemplary methods, oat hull processing can include removal of undesirable components, for example, by subjecting oat hulls to warm water washes and/or to chemical-based treatments (e.g., sodium hydroxide and heat) prior to cooking. Undesirable compounds for removal or reduction, can include, but are not limited to, inorganic compounds, sulfur-containing compounds, chloride, sodium, phosphorus, magnesium, starches and the like. For example, oat hulls can be treated with warm water washes with continuous mixing and the application of indirect heat (e.g., about 50° C.) for about 1 hour, after which the liquid that contains the undesirable components can be drained or decanted, with or without centrifugation, to obtain about a 60% dewatered mixture. In some methods, these warm water washes can reduce the amount of undesirable components in subsequent extracts by at least about 25%, or at least about 50%, as illustrated below in Table 6 (See the lower levels of undesirable components in the washed oat hull sample, e.g., “Warm Water Washed XOS,” compared to the non-water washed control, e.g., “Wetted XOS). Warm water washes can be effective for reducing amounts of chlorine, sulfate, potassium, magnesium and phosphorus, as well as other undesirable agents and for increasing the overall recovery of xylan in a desirable extract (e.g. of use as a consumable).

TABLE 6
Characterization of undesirable components in oat hull extracts

| | Total | | | | | | | | | | | |
|---|---|---|---|---|---|---|---|---|---|---|---|---|
| | Solids | Ash | Cl | SO4 | Ca | Fe | K | Mg | Na | P | Zn | Si |
| | % w/w | % w/w | ppm | ppm | ppm | ppm | ppm | ppm | ppm | ppm | ppm | ppm |
| Warm Water Washed XOS | 8.8 | 0.2 | 95.0 | 25.7 | 263.7 | 0.9 | 531.3 | 205.3 | 57.0 | 213.3 | 2.2 | 78.0 |
| Wetted XOS (control) | 9.7 | 0.5 | 968.3 | 98.3 | 255.3 | 1.4 | 2553.3 | 381.7 | 78.3 | 448.3 | 2.8 | 79.3 |

All of the COMPOSITIONS and METHODS disclosed and claimed herein can be made and executed without undue experimentation in light of the present disclosure. While the compositions and methods have been described in terms of preferred embodiments, it is apparent to those of skill in the art that variations maybe applied to the COMPOSITIONS and METHODS and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. A xylo-oligosaccharide-rich extract composition derived from oat hulls, the composition comprising:
xylo-oligosaccharides with degrees of polymerization (DP) 3 or greater present in the xylo-oligosaccharide-rich extract at a concentration equal to or greater than the concentration of DP2 and DP1 xylans, wherein the amount of the xylo-oligosaccharides with DP3 or greater present in the xylo-oligosaccharide-rich extract is at least 15% of total xylan present in the oat hulls.

2. The composition of claim 1, wherein the concentration of xylo-oligosaccharides with DP3 or greater compared to the concentration of DP2 and DP1 is at least a 1 to 1 ratio.

3. The composition of claim 1, wherein the concentration of xylo-oligosaccharides with DP3 or greater compared to the concentration of DP2 and DP1 is at least a 1.5 to 1 ratio.

4. The composition of claim 1, wherein the concentration of xylo-oligosaccharides with DP3 or greater compared to the concentration of DP2 and DP1 is at least a 2 to 1 ratio.

5. The composition of claim 1, wherein the concentration of xylo-oligosaccharides with DP3 or greater compared to the concentration of DP2 and DP1 is at least a 3 to 1 ratio.

6. The composition of claim 1, wherein the amount of xylo-oligosaccharides with DP3 or greater in the xylo-oligosaccharide-rich extract is 20.0% to 80.0% of the total xylan present in the oat hulls.

7. The composition of claim 1, wherein the amount of xylo-oligosaccharides with DP3 or greater in the xylo-oligosaccharide-rich extract is 25.0% to 70.0% of the total xylan present in the oat hulls.

8. The composition of claim 1, wherein the amount of xylo-oligosaccharides with DP3 or greater in the xylo-oligosaccharide-rich extract is 30.0% to 60.0% of the total xylan present in the oat hulls.

9. The composition of claim 1, wherein levels of chloride, sulfate, and sodium have been reduced in the xylo-oligosaccharide-rich extract composition compared to an xylo-oligosaccharide-rich extract prepared from an unprocessed oat hull byproduct.

10. The composition of claim 1, wherein the xylo-oligosaccharide-rich extract composition comprises an ingredient in a food product.

11. The composition of claim 1, wherein the xylo-oligosaccharide-rich extract composition is formulated as a fiber supplement.

12. A method for obtaining xylo-oligosaccharide-rich extracts from oat hulls enriched with degrees of polymerization (DP) 3 or greater xylo-oligosaccharides, the method comprising:
providing oat hulls;
treating the oat hulls to remove undesirable compounds;
cooking the oat hulls at a temperature between about 170° C. to about 220° C. for about 1 minute to about 30 minutes; and
separating an insoluble solids fraction from a soluble solids fraction, the soluble solids fraction comprising an xylo-oligosaccharide-rich extract comprising enriched xylo-oligosaccharides with DP3 or greater.

13. The method of claim 12, wherein the undesirable compounds comprise inorganic compounds.

14. The method of claim 12, wherein the undesirable compounds comprise one or more of sulfate, sulfur-containing compounds, chloride, sodium, phosphorus, and magnesium.

15. The method of claim 12, wherein the undesirable compounds comprise starches.

16. The method of claim 12, wherein the oat hulls are milled prior to cooking.

17. The method of claim 12, wherein treating the oat hulls comprises washing the oat hulls with an aqueous solution and draining off the aqueous solution to remove undesirable compounds.

18. The method of claim 12, wherein the oat hulls are diluted in an aqueous solution after cooking the oat hulls.

19. The method of claim 12, wherein the insoluble solids fraction is washed prior to being subject to additional cooking and separating to obtain remaining xylo-oligosaccharides with DP3 or greater.

20. The method of claim 12, wherein the oat hulls are cooked at a temperature of about 180° C. to about 210° C.

21. The method of claim 12, wherein the oat hulls are cooked at a temperature of about 185° C. to about 195° C.

22. The method of claim 12, wherein the oat hulls are cooked for about 2 minutes to about 20 minutes.

23. The method of claim 12, wherein the oat hulls are cooked for about 3 minutes to about 10 minutes.

24. The method of claim 12, wherein the xylo-oligosaccharides with DP3 or greater are present in the xylo-oligosaccharide extract at a concentration equal to or greater than the concentration of xylans with DP2 and DP1, and wherein the amount of the xylo-oligosaccharides with DP3 or greater in the xylo-oligosaccharide-rich extract is at least 15% of total xylan present in the oat hulls.

25. The method of claim 24, wherein the concentration of xylo-oligosaccharides with DP3 or greater compared to the concentration of DP2 and DP1 is at least a 1 to 1 ratio.

26. The method of claim 24, wherein the concentration of xylo-oligosaccharides with DP3 or greater compared to the concentration of DP2 and DP1 is at least a 1.5 to 1 ratio.

27. The method of claim 24, wherein the concentration of xylo-oligosaccharides with DP3 or greater compared to the concentration of DP2 and DP1 is at least a 2 to 1 ratio.

28. The method of claim 24, wherein the concentration of xylo-oligosaccharides with DP3 or greater compared to the concentration of DP2 and DP1 is at least a 3 to 1 ratio.

29. The method of claim 24, wherein the amount of xylo-oligosaccharides with DP3 or greater in the xylo-oligosaccharide extract is 20.0% to 80.0% of the total xylan present in the oat hulls.

30. The method of claim 24, wherein the amount of xylo-oligosaccharides with DP3 or greater is 25.0% to 70.0% of the total xylan present in the oat hulls.

31. The method of claim 24, wherein the amount of xylo-oligosaccharides with DP3 or greater is 30.0% to 60.0% of the total xylan present in the oat hulls.

32. The method of claims 24, further comprising lightening the color of the xylo-oligosaccharide-rich extract.

33. The method of claim 24, further comprising forming a powder from the xylo-oligosaccharide-rich extract.

34. The method of claim 24, further comprising adding additional agents to the xylo-oligosaccharide-rich extract prior to adding the xylo-oligosaccharide-rich extract to a consumable food product.
